(19) United States
(12) Patent Application Publication
(10) Pub. No.: US 2006/0135967 A1
(43) Pub. Date: Jun. 22, 2006

(54) Method and apparatus for attaching a valve prosthesis

(76) Inventor: Fidel Realyvasquez, Palo Cedro, CA (US)

Correspondence Address: HELLER EHRMAN LLP, 275 MIDDLEFIELD ROAD, MENLO PARK, CA, 94025-3506, US

(21) Appl. No.: 11/286,318

(22) Filed: Nov. 22, 2005

Related U.S. Application Data
(60) Provisional application No. 60/629,983, filed on Nov. 22, 2004.

(51) Int. Cl. A61B 17/10

(52) U.S. Cl. 606/142

(57) ABSTRACT

A valve delivery device and method of use is provided. In one embodiment, the device to deliver a valve prosthesis to a target tissue may include at least one anchor and at least one guide wire coupled to the anchor. The device has a fastener housing. The device may also include a first set of fasteners in said fastener housing, wherein the fasteners are movable from a first position to a second, tissue engagement position. The device may also include a second set of fasteners housed in the anchor to attach the anchor to the tissue, wherein the fasteners are movable from a first position to a second, tissue engagement position. The guide wire may be slidably received in the fastener housing and has a length sufficient to extend from the fastener housing to the target tissue site, wherein the guide wire is used to direct the fastener housing into place.

This application claims the benefit of priority from U.S. Provisional Application Ser. No. 60/629,983 (Attorney Docket Number 40450-0010US) filed on Nov. 22, 2004, fully incorporated herein by reference for all purposes.

### BACKGROUND OF THE INVENTION

1. Technical Field

The invention relates to apparatus and methods for prosthesis implantation and is especially useful in aortic valve repair procedures.

2. Background Art

Essential to normal heart function are four heart valves, which allow blood to pass through the four chambers of the heart in one direction. The valves have either two or three cusps, flaps, or leaflets, which comprise fibrous tissue that attaches to the walls of the heart. The cusps open when the blood flow is flowing correctly and then close to form a tight seal to prevent backflow.

The four chambers are known as the right and left atria (upper chambers) and right and left ventricles (lower chambers). The four valves that control blood flow are known as the tricuspid, mitral, pulmonary, and aortic valves. In a normally functioning heart, the tricuspid valve allows one-way flow of deoxygenated blood from the right upper chamber (right atrium) to the right lower chamber (right ventricle). When the right ventricle contracts, the pulmonary valve allows one-way blood flow from the right ventricle to the pulmonary artery, which carries the deoxygenated blood to the lungs. The mitral valve, also a one-way valve, allows oxygenated blood, which has returned to the left upper chamber (left atrium), to flow to the left lower chamber (left ventricle). When the left ventricle contracts, the oxygenated blood is pumped through the aortic valve to the aorta.

Certain heart abnormalities result from heart valve defects, such as valvular insufficiency. Valve insufficiency is a common cardiac abnormality where the valve leaflets do not completely close. This allows regurgitation (i.e., backward leakage of blood at a heart valve). Such regurgitation requires the heart to work harder as it must pump both the regular volume of blood and the blood that has regurgitated. Obviously, if this insufficiency is not corrected, the added workload can eventually result in heart failure.

Another valve defect or disease, which typically occurs in the aortic valve is stenosis or calcification. This involves calcium buildup in the valve which impedes proper valve leaflet movement.

In the case of aortic valve insufficiency or stenosis, treatment typically involves removal of the leaflets and replacement with valve prosthesis. However, known procedures have involved generally complicated approaches that can result in the patent being on cardio-pulmonary bypass for an extended period of time.

Applicants believe that there remains a need for improved valvular repair apparatus and methods that use minimally invasive techniques and/or reduce time in surgery.

### SUMMARY OF THE INVENTION

The present invention involves valve repair apparatus and methods that overcome problems and disadvantages of the prior art. The present invention may facilitate the delivery and attachment of various prosthetic device into the body. The present invention may also reduce the amount time used to perform a delivery and attachment procedure.

In one aspect of the present invention, a method for attaching a valve prosthesis to a target tissue is provided. The method includes attaching a plurality of anchors each having a guide wire to the target tissue. A delivery device having a fastener housing may be slidably advanced along the guide wires, wherein the fastener housing has grooves or slots for receiving the guide wires. The method may include positioning the delivery device so that fasteners inside the fastener housing will engage the target tissue when the fasteners are deployed. The guide wires may be clipped so that the anchors can remain embedded in tissue when the guide wires are removed.

The method may also include sliding the fastener so that a sewing ring on the valve prosthesis is advanced over barbed portion of the anchor. A second set of fasteners may be deployed with each of the fasteners housed in the anchors. The fasteners may be delivered along a curved path near the distal en of the barbed portion of the anchor. The fasteners may assume a first linear configuration and a second curled configuration once delivered at the target tissue. A proximal end of each of the fasteners may extend outward from an opening on the anchor to secure the fastener to the anchor. The method may include removing the delivery device and leaving the plurality of fasteners with the valve prosthetic attached to the target tissue. A hollow sharpened shaft may be extended from the fastener housing to pierce through a sewing ring on the valve prosthesis. The method may include advancing a push rod through the sharpened shaft to extend a fastener outward from the hollow shaft. The target tissue may be an aortic valve annulus with valve leaflets removed. The valve prosthesis may be a mechanical valve.

In another aspect of the present invention, a device for use with a valve prosthesis is provided. The device to deliver a valve prosthesis to a target tissue may include at least one anchor and at least one guide wire coupled to the anchor. The device may have a fastener housing. The device may also include a first set of fasteners in said fastener housing, wherein the fasteners are movable from a first position to a second, tissue engagement position. The device may also include a second set of fasteners housed in the anchor to attach the anchor to the tissue, wherein the fasteners are movable from a first position to a second, tissue engagement position. The guide wire may be slidably received in the fastener housing and has a length sufficient to extend from the fastener housing to the target tissue site, wherein the guide wire is used to direct the fastener housing into place.

The device may also include an anchor having a sharpened barb portion for attaching the anchor to a sewing ring on the valve prosthesis. The fasteners may have a curved distal portion, a sharpened distal tip, or a blunt distal tip. The fasteners may be made of a shape memory material such as but not limited to nitinol or the like.

The above is a brief description of some deficiencies in the prior art and advantages of the present invention. Other features, advantages, and embodiments of the invention will be apparent to those skilled in the art from the following description and accompanying drawings, wherein, for purposes of illustration only, specific forms of the invention are set forth in detail. A further understanding of the nature and advantages of the invention will become apparent by reference to the remaining portions of the specification and drawings.

A further understanding of the nature and advantages of the invention will become apparent by reference to the remaining portions of the specification and drawings.

### BRIEF DESCRIPTION OF THE DRAWINGS

FIG. 1 is a perspective view of a valve prosthesis being lowered to a target tissue.

FIGS. 2A-2C show various views of the embodiments of the present invention.

FIGS. 3A-3B are various views showing the delivery of a fastener according to the present invention.

FIGS. 4A-4B are various views showing the delivery of a fastener according to the present invention.

FIG. 5 shows a perspective view of a mechanical valve fastened to target tissue.

FIGS. 6A-6B shows delivery of a fastener.

FIG. 7 shows delivery of a fastener.

FIGS. 8A-9 show close-up views of a delivery of a fastener.

FIG. 10 shows one embodiment of a kit according to the present invention.

### DESCRIPTION OF THE SPECIFIC EMBODIMENTS

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

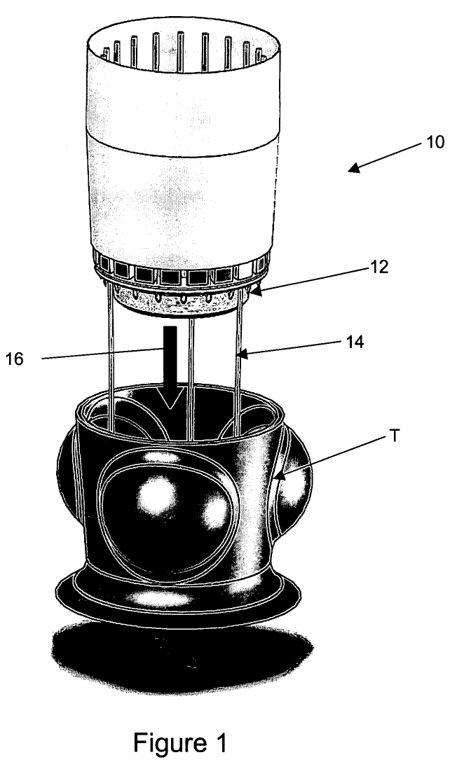

Referring now to FIG. 1, one embodiment of the present invention will now be described. FIG. 1 shows a portion of aortic tissue T and a portion of delivery device 10 for attaching a mechanical valve prosthesis 12 at a target site inside the tissue T. The delivery device 10 with a fastener housing is guided to the target site by at least one guide wire 14. The delivery device 10 will be advanced towards the target site in the direction indicated by arrow 16. The guide wire 14 facilitates the placement and attachment of the valve prosthesis since the guide wire 14 is anchored at and/or near the target tissue.

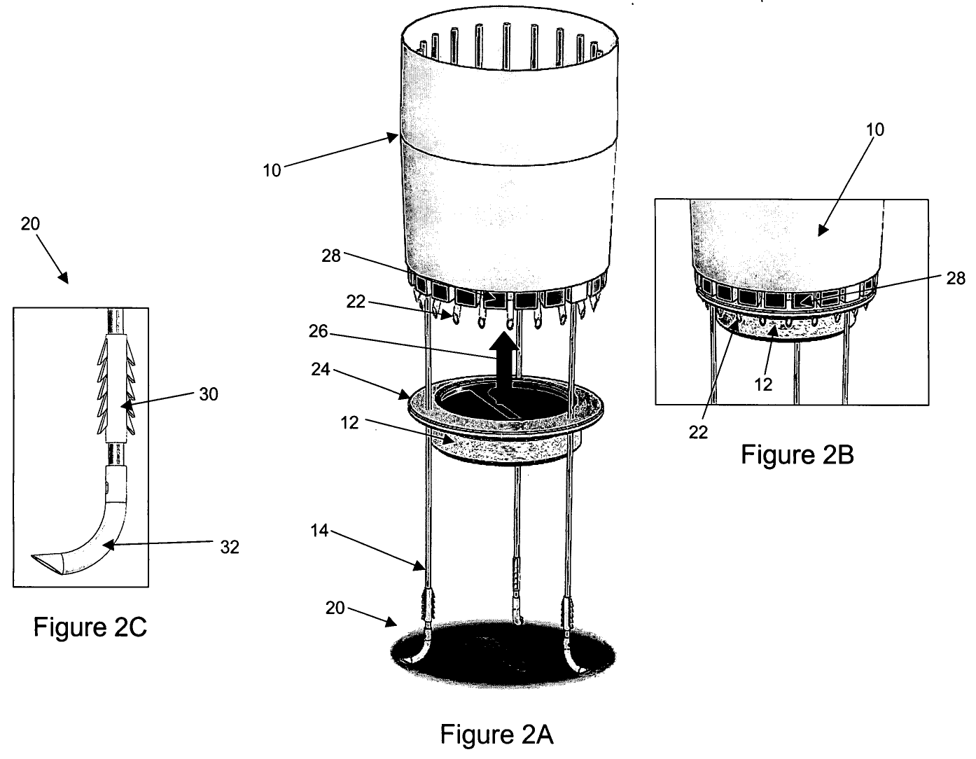

FIG. 2A shows the delivery device 10 with the aortic tissue T removed to more clearly show the guide wires 14 and the anchors 20. In this present embodiment, the delivery device 10 may be tethered to three anchors 20 via the guide wires 14. The user secures anchors 20 to the target tissue. The delivery device 10 and the valve prosthesis mounted thereon are guided into position by following the guide wires 14 coupled to the anchors 20. In this present embodiment, the delivery device 10 is loaded with 20 fasteners inside guide shafts 22. Once the device 10 is guided into position by following the guide wires 14 coupled to the anchors 20, the user then deploys fasteners through the guide shafts 22 via the delivery device 10.

As more clearly shown in FIG. 2B, the mounting of the mechanical valve 12 on the delivery device 10 will now be described. The mechanical valve 12 seats into device 10. In the present embodiment, the mechanical valve 12 has a sewing ring 24 through which the guide shafts 22 penetrate. As the mechanical valve 12 is seated, the sharpened tip of the shafts 22 will penetrate the sewing ring 24 as the valve is placed in the delivery device 10 as indicated by arrow 26 (shown in FIG. 2A). In the present embodiment, the mechanical stops 28 on delivery device 10 will contact the mechanical valve prosthesis 12 when the prosthesis is properly mounted on the device 10.

FIG. 2C shows a close up view of the anchor 20. In the present embodiment,

the anchor 20 may include a barbed portion 30 and a curved, sharpened portion 32. As will be discussed later, the barbed portion 30 have barbs or teeth oriented to allow the sewing ring 24 to slide down into position but prevent the sewing ring from pulling off the barbed portion 30 when retracted. The curved portion 32 with the sharpened tip will act as anchors in tissue in the annulus. Fasteners may be housed inside the portion 32 and pushed out by pushers (hidden in this view) inside the guide wire 14.

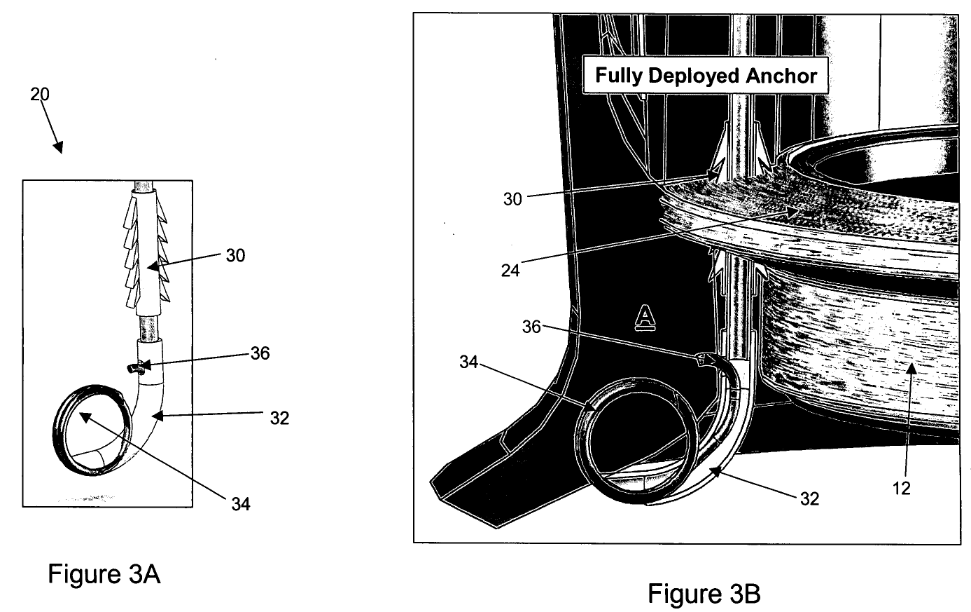

Referring now to FIGS. 3A and 3B, the deployment of the valve prosthesis 12 over the anchor 20 will now be described. FIG. 3A shows that a fastener 34 is deployed from the anchor 20 to secure the anchor to tissue. Deployment causes a fastener tip to 36 catch into hole on the curved portion 32, securing the fastener 34 to the curved portion 32 and thus the portion 32 to the tissue.

FIG. 3B is a cross-sectional view of the anchor 20 when the valve prosthesis 12 is mounted over the barbed portion 30. As seen, the aortic tissue at the annulus A is targeted. Barbs capture and hook into sewing ring 24 on the mechanical valve prosthesis 12 as the prosthesis is lowered over the portion 30. This will secure the mechanical valve prosthesis 12 to the anchor 20 prior to the deployment of additional fasteners through the sewing ring 24 which further secure the prosthesis 12 to the annulus A.

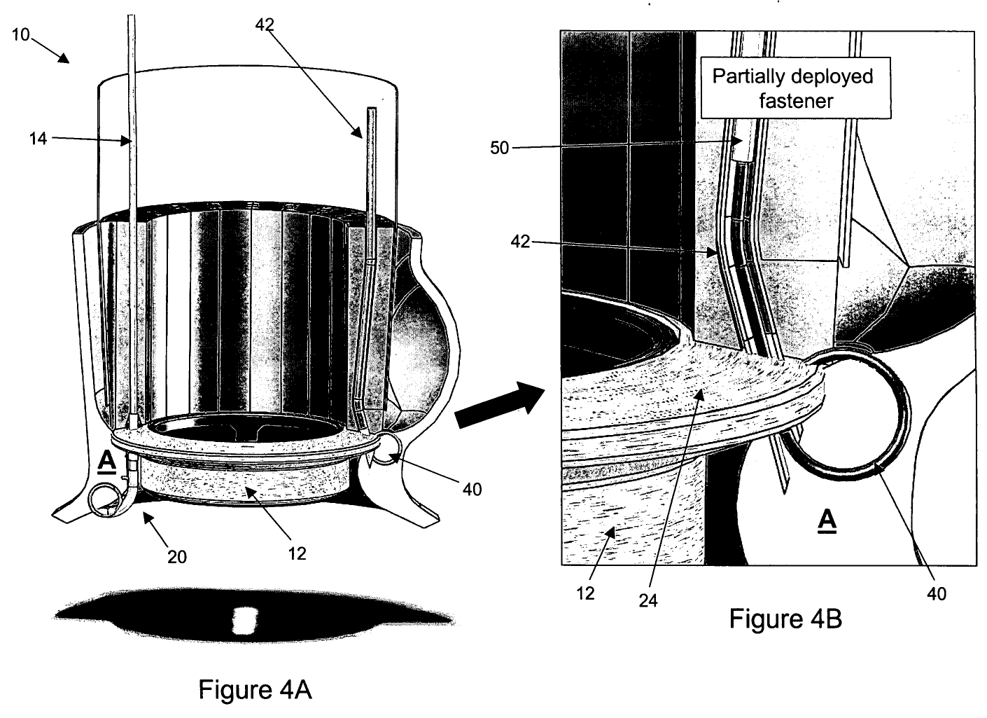

Referring now to FIGS. 4A and 4B, deployment of additional fasteners to attach the valve prosthesis 12 to the target tissue at the annulus A will now be described. FIG. 4A shows that once the delivery device 10 is delivered down the guide wire 14, a plurality of additional fasteners 40 may be deployed to attached the sewing ring 24 to the tissue at annulus A. A flexible shaft 42 having a sharpened tip may be slidably extended to from the delivery device 10 to piece the sewing ring 24 and then allow the fasteners 40 to be deployed. The fastener 40 will pierce into the tissue at annulus A and then curl back to pierce into the sewing ring 24.

As seen in FIG. 4B, the fastener 40 is deployed by using a pusher 50 which will slide inside the shaft 42. As the fastener 40 extends outward, its shape memory material will begin to curl the fastener back towards the sewing ring 24. The shaft 42 is shaped so that there is a bend or a curve near the distal, sharpened end. This will orient the fasteners 40 to extend outward from the shaft 42 and curl back towards to sewing ring 24 to penetrate the ring and secure it to the tissue.

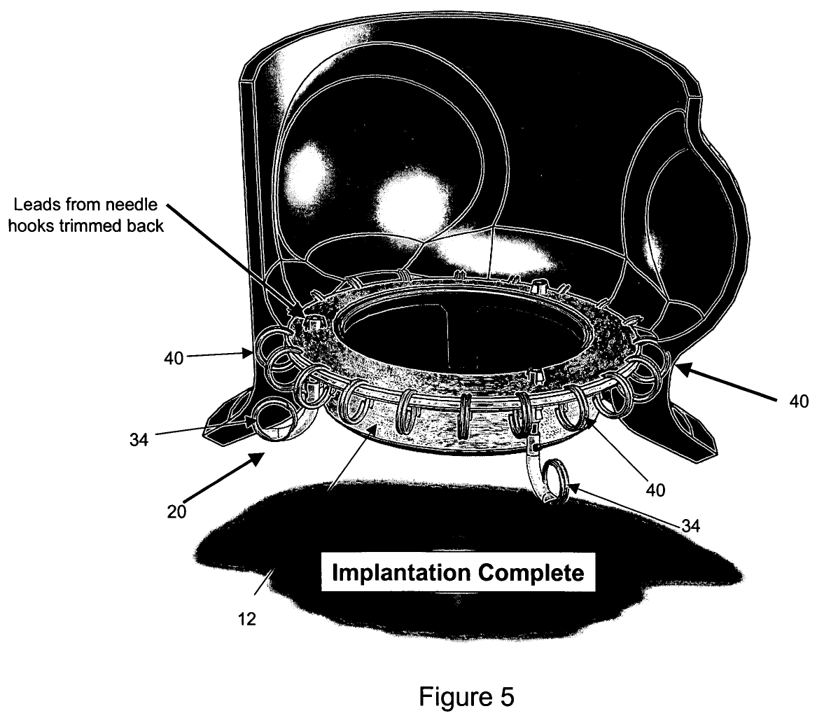

FIG. 5 shows the mechanical valve prosthesis 12 after implantation is completed, with about twenty fasteners 40 attached to the sewing ring 24. The anchors 20 which were attached to the guide wires 14 to direct the delivery device 10 in place will remain attached to the tissue and also attached to the sewing ring 24. The guide wires 14 may be trimmed off so that they may be removed while leaving the anchors 20 behind. The fasteners 34 will hold the anchors 20 to the tissue and the barbs on the portion 30 will hold the anchors 20 to the sewing ring. This will further improve the fastening of the mechanical valve prosthesis 12 to the tissue at annulus A.

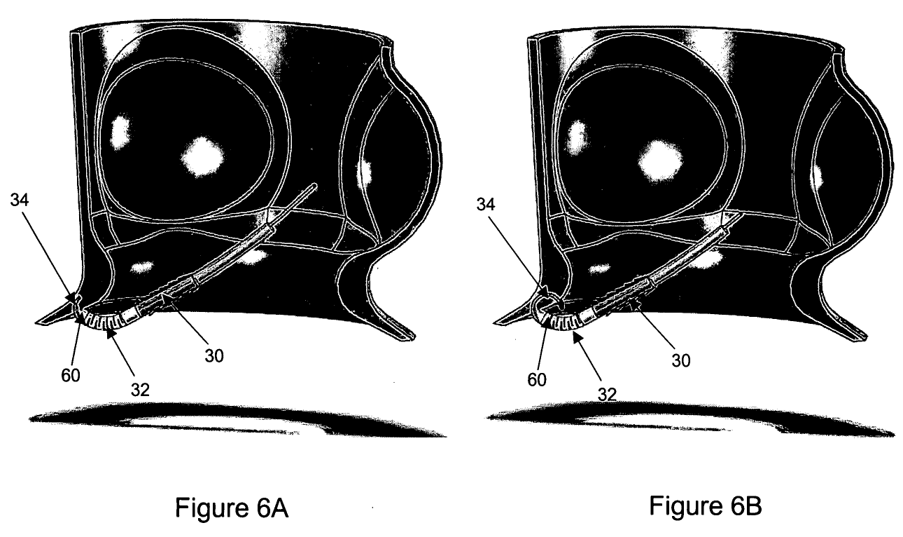

Referring now to FIGS. 6A and 6B, one embodiment of the method and apparatus for attaching anchors 20 prior to deployment of delivery device 10 will now be described. FIG. 6A shows that the sharpened tip 60 of portion 32 is pierced into the tissue. Fastener 34 also begins to pierce the tissue. FIG. 6B shows that the fastener 34 will extend outward from portion 32 and begin to curl.

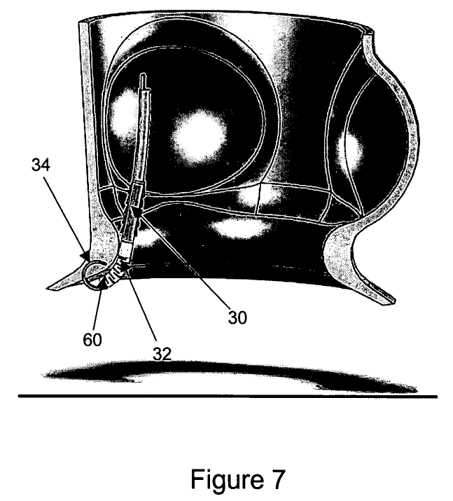

FIG. 7 shows that the sharpened portion 32 may be moved to a more vertical orientation in preparation to guiding the delivery device 10 in place. By way of example and not limitation, the sharpened tip 60 may be retracted out of the tissue.

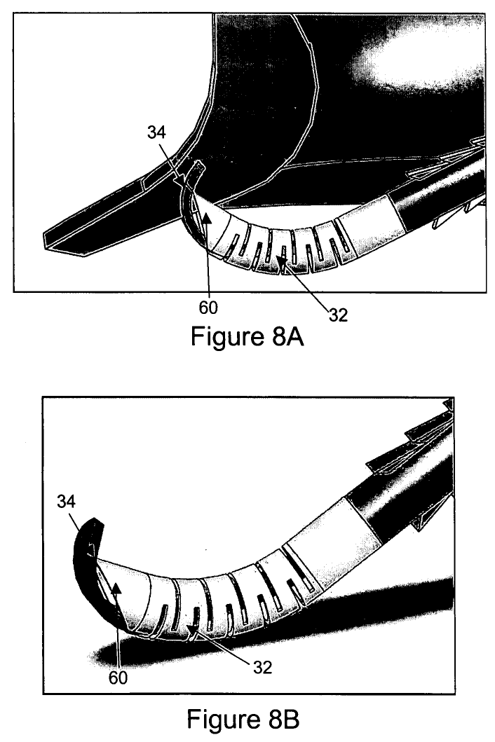

FIGS. 8A and 8B show close up views of the sharpened tip 60 and the portion 32. The portion 32 may be laser cut to have a plurality of slots to facilitate the shaping of the portion 32.

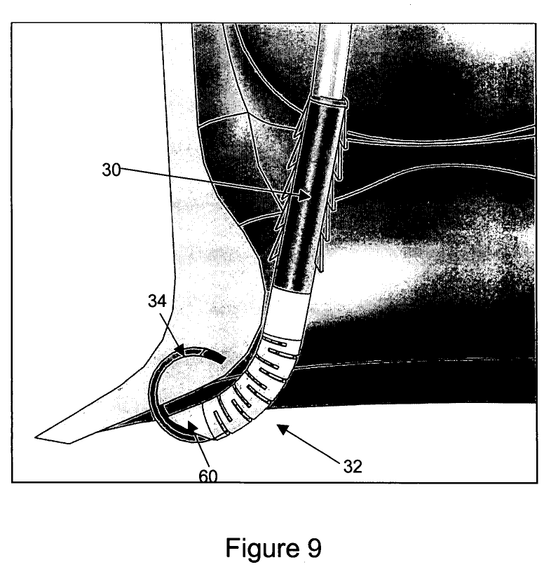

FIG. 9 shows a close up view of the portion 32 with the sharpened tip 60 retracted and the portions 32 and 30 positioned more vertically to guide the device 10 into position.

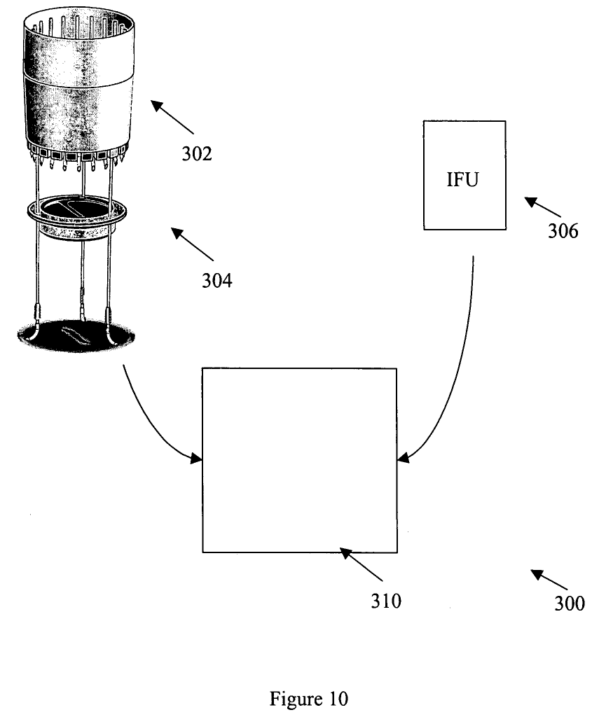

Referring now to FIG. 10, a kit 300 according to the present invention will be shown. The kit 300 may comprise of a valve prosthesis delivery device 302, a valve prosthesis 304, and instructions for use (IFU) 306 setting forth the method of delivery or attachment. The kit may also include a container 310 sized to house the valve prosthesis delivery device, the valve prosthesis, and the instructions for use. In some embodiments, the prosthesis 304 may be separate from the kit.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, a prosthetic valve or a graft may be premounted on to the apparatus. With any of the above embodiments, the apparatus may be configured to be delivered percutaneously or through open surgery. Some of the embodiments above may not use a barbed portion 32. The number of fasteners used may also be varied. By way of example and not limitation, some embodiments may only have 3-5 fasteners. Others may have more than 10, 15, 20, 25, 30, 25, 40, 45, 50, or more fasteners per sewing ring. It should be understood that the present invention may also be used with non-mechanical valves that have a sewing ring or penetratable outer ring that the fasteners may pierce to secure the valve to target tissue.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A device to deliver a valve prosthesis to a target tissue, the device comprising:
at least one anchor;
at least one guide wire coupled to the anchor;
a fastener housing;
a first set of fasteners in said fastener housing, wherein said fasteners are movable from a first position to a second, tissue engagement position; and
a second set of fasteners housed in said anchor to attach the anchor to the tissue, wherein the fasteners are movable from a first position to a second, tissue engagement position;
wherein said guide wire is slidably received in the fastener housing and has a length sufficient to extend from the fastener housing to the target tissue site, said guide wire used to direct the fastener housing into place.

2. The device of claim 1 wherein the system further comprises a plunger member, wherein the plunger member is movable along a longitudinal axis of the device and has a plurality of push rods sized sufficient to move said fasteners from a first position to a second, tissue engagement position.

3. The device of claim 1 wherein anchor includes a sharpened barb portion for attaching the anchor to a sewing ring on the valve prosthesis.

4. The device of claim 1 wherein the fasteners have a curved distal portion.

5. The device of claim 1 wherein the fasteners have a sharpened distal tip.

6. The device of claim 1 wherein the fasteners have a blunt distal tip.

7. The device of claim 1 wherein the fasteners are made of a shape memory material.

8. The device of claim 1 wherein the valve prosthesis is a mechanical valve

9. A method for attaching a valve prosthesis to a target tissue, the method comprising:
attaching a plurality of anchors each having a guide wire to the target tissue;
slidably advancing a delivery device having a fastener housing along the guide wires, wherein said fastener housing has grooves or slots for receiving the guide wires;
positioning the delivery device so that fasteners inside the fastener housing will engage the target tissue when the fasteners are deployed; and
clipping the guide wires so that the anchors can remain embedded in tissue when the guide wires are removed.

10. The method of claim 9 further comprising sliding the fastener so that a sewing ring on the valve prosthesis is advanced over barbed portion of the anchor.

11. The method of claim 9 further comprising deploying a second set of fasteners, each of the fasteners housed in the anchors.

12. The method of claim 9 wherein the fasteners are delivered along a curved path near the distal en of the barbed portion of the anchor.

13. The method of claim 9 wherein the fasteners assume a first linear configuration and a second curled configuration once delivered at the target tissue.

14. The method of claim 9 further comprising deploying a second set of fasteners, each of the fasteners housed in the anchors;
wherein a proximal end of each of the fasteners extends outward from an opening on the anchor to secure the fastener to the anchor.

15. The method of claim 9 further comprising removing said delivery device and leaving the plurality of fasteners with the valve prosthetic attached to the target tissue.

16. The method of claim 9 further comprising extending a hollow sharpened shaft from the fastener housing to pierce through a sewing ring on the valve prosthesis;
advancing a push rod through the sharpened shaft to extend a fastener outward from the hollow shaft.

17. The method of claim 9 wherein the target tissue is an aortic valve annulus with valve leaflets removed.

18. The method of claim 9 wherein the valve prosthesis is a mechanical valve.

19. A kit comprising:
a valve prosthesis delivery device;
a valve prosthesis;
instructions for use setting forth the method of claim 9; and
a container sized to house the valve prosthesis delivery device, the valve prosthesis, and the instructions for use.
